(19) United States
(12) Patent Application Publication
(10) Pub. No.: US 2017/0283160 A1
(43) Pub. Date: Oct. 5, 2017

(54) SEA SALT MOUTH RINSE KIT AND METHOD OF USING SAME

(71) Applicant: SEA SALT SOLUTIONZ, Brampton (CA)

(72) Inventor: Cathryn Elizabeth ALLEN, Brampton (CA)

(21) Appl. No.: 15/471,533

(22) Filed: Mar. 28, 2017

Related U.S. Application Data
(60) Provisional application No. 62/314,471, filed on Mar. 29, 2016.

(51) Int. Cl. B65D 85/00; A61K 33/00; B65D 41/02; B65D 75/58; B65D 1/02; B65D 25/56

(52) U.S. Cl. CPC B65D 85/70; B65D 1/02; B65D 25/56; B65D 41/02; B65D 75/58; A61K 33/00

(57) ABSTRACT

A sea salt mouth rinse kit includes: a bottle with a fill line; and a plurality of packets, each containing a predetermined quantity of pure sea salt. In a water filling step, a user fills the bottle with water to the fill line, such that it then contains a predetermined volume of water, at a tepid or room temperature. In a salt adding step, the user empties one of the packets into the bottle. The predetermined quantity of pure sea salt fully dissolves and closely approaches full saturation, without precipitating out, in the predetermined volume of water at a water pressure and pH both falling within normal ranges for distilled water consumed by humans. A user selectively rinses with the water through an opening in the bottle, and a cap selectively securely closes and seals the opening.

### FIELD

The invention relates generally to the field of mouth rinse kits and, more particularly, to a sea salt mouth rinse kit and method of using same.

### INTRODUCTION

Warm salt water rinses may be known, in the prior art, to have various advantageous utilities. It was said that Hippocrates, a notional father of modern medicine, made use of remedies containing salt after noticing the therapeutic qualities of seawater.

Today, many millions of cancer cases may exist around the world. Millions more new cancer cases may be diagnosed each year, and many thousands of people may die of cancer each day. These numbers may be expected only to rise going forward. Oral mucositis may be a common, debilitating complication of cancer treatments, particularly chemotherapy and radiation, which can cause pain, restrict oral intake, act as a portal of entry for organisms, contribute to interruption of therapy, require an increased use of antibiotics and narcotics, increase the length of hospitalization, and/or increase the overall cost of treatment. Starting a regimen of rinsing with salt water, before beginning radiation or chemotherapy treatment, may help alleviate the side effects and symptoms or oral mucositis.

Also, mouth ulcers may be caused by an underlying medical condition, such as, coeliac disease, Crohn's disease, reactive arthritis, immunodeficiencies, gastrointestinal diseases, a sore throat, and/or hand, foot and mouth disease. Additionally, mouth sores may be associated with systemic lupus erythematosus and/or Behcet's disease as well.

Gargling with salt water may help with mouth ulcers, mouth sores, canker sores, sore throat, reducing painful swelling of cells in the mucous membrane at the back of the throat, and/or keep the area clean. Salt water may have astringent qualities and/or may help to speed wound healing, possibly by reducing inflammation and/or contracting the tissues. As an astringent, dentists, orthodontists, oral surgeons, or periodontists may use warm salt water rinses in an effort to promote wound healing by reducing inflammation and contracting the tissues, and to ease pain.

In the throat, salt water may draw moisture out of any bacteria there, and out of the throat's swollen tissues relieving inflammation. A salty environment may prevent bacteria from growing. Also, though not essential to the working of the invention, some may believe that salt water rinses may help to alkalinize the mouth and that the alkalinity may help to decrease bacteria count. There may also be some benefit to using pure salt with a natural chemical structure in this respect, rather than using a table salt which may have been altered through processing.

### SUMMARY

Embodiments described herein provide a sea salt mouth rinse kit and method of using same that may be beneficial in many applications, potentially including one or more of the following, among others:
During chemotherapy—inhibit formation or minimize or reduce a chance of forming blisters in the mouth (which can be a very painful, and debilitating side effect during chemotherapy).
After dental work, oral surgery, orthodontic work, periodontal work, among others—to help soothe the mucosal tissue and keep the surgical site clean.
For sore throats—gargling with the solution may help to reduce inflammation and/or reduce pain.

According to some embodiments, the sea salt mouth rinse kit and method of using same, offers simple and convenient advantages to people experiencing extreme health issues. Vulnerable people, such as those who have taken ill, may find that such simplicity can help them on their healing journey.

By way of illustrative example, the inventor herein was undergoing extensive dental work (which included bone grafting and dental implants). She then consistently found it easy and convenient to use her invention, many times throughout the day, over the course of her healing process. When later diagnosed with cancer, the inventor was apprised of a very real possibility that her bone graft would not survive the chemotherapy. Even then, she found that her invention facilitated continued and extensive use of sea salt mouth rinses throughout her chemotherapy treatments as a preventative measure against the formation of mouth blisters (which can be a very painful side effect of chemotherapy). To the inventor's surprise, her mouth remained blister free, and the bone graft remained intact and healthy enough to sustain dental implants at a further date. Since then, the inventor has found it simple and convenient to make further use of her invention for sea salt mouth rinses, among other things, to soothe a sore throat when the need has arisen.

In an aspect, embodiments described herein provide a mouth rinse kit or a sea salt mouth rinse kit, and/or a method of using same.

In an aspect, embodiments described herein can help inhibit formation or minimize or reduce a chance of forming blisters in the mouth, to help soothe mucosal tissue, to help keep dental surgical sites clean, to help soothe a sore throat, and/or to help to reduce inflammation and/or reduce pain. In an aspect, embodiments described herein can help increase the chances that dental bone grafts might survive chemotherapy intact and remain healthy enough to thereafter sustain dental implants.

In an aspect, embodiments described herein can help people rinse the mouth with sea salt many times throughout the day. In an aspect, embodiments described herein can help provide an easy, simple, ready and/or convenient way to rinse the mouth with a sea salt solution. In an aspect, embodiments described herein can help people experiencing health issues and/or otherwise vulnerable people rinse their mouths with sea salt.

In an aspect, embodiments described herein provide a sea salt mouth rinse kit which includes a water container and one or more sea salt containers. The water container has a fill line and is adapted to contain a predetermined volume of water when filled to the fill line. Each of the sea salt containers contains a predetermined quantity of a sea salt material. According to the invention, the predetermined quantity of the sea salt material is adapted to fully dissolve to a predetermined saturation, without precipitating out, in the predetermined volume of water at a predetermined temperature, and at a predetermined water pressure and pH. The predetermined temperature is a water temperature comfortably consumed by humans, and the predetermined water pressure and pH both fall within ranges consumed by humans.

In an aspect, in some embodiments each of the sea salt containers is a closed packet which may be selectively torn open, and the sea salt material is pure sea salt.

In some embodiments, the water container comprises a lid and the water container is shaped to define an opening. The opening can be adapted to permit a user to selectively pour or rinse with the predetermined volume of water therethrough when the container is filled. The lid can be selectively used to securely close and seal the opening.

According to some embodiments, the predetermined saturation closely approaches full saturation.

According to some embodiments, the predetermined temperature falls within a temperature range of between about tepid temperature and about room temperature.

According to some embodiments, the predetermined volume of water falls within a volume range of between about 0.18 liters (6 ounces) and about 1.5 liters (50.7 ounces) and, more preferably, between about 0.40 liters (13.5 ounces) and about 0.75 liters (25.4 ounces). The predetermined volume of water may be between about 0.41 liters (14.0 ounces) and about 0.47 liters (16.0 ounces).

According to some embodiments, the predetermined water pressure and pH may fall within normal ranges for distilled and tap waters consumed by humans.

According to some embodiments, the water container is a bottle, and the lid is a cap.

According to some embodiments, there is also disclosed a method of using a sea salt mouth rinse kit which includes a water filling step and a salt adding step. The sea salt mouth rinse kit is of a type which includes a water container and one or more sea salt containers. The water container is of a type which has a fill line. Each of the sea salt containers is of a type which contains a predetermined quantity of a sea salt material. According to the invention, in the water filling step, a user fills the water container with water to the fill line, such that the water container then contains a predetermined volume of water. In the water filling step, the water is at a predetermined temperature, and at a predetermined water pressure and pH. In the water filling step, the predetermined temperature is a water temperature comfortably consumed by humans, and the predetermined water pressure and pH both fall within ranges consumed by humans. In the salt adding step, the user opens and empties one of the sea salt containers into the water container, such that the predetermined quantity of the sea salt material fully dissolves to a predetermined saturation, without precipitating out, in the predetermined volume of water.

According to some embodiments, in the salt adding step, each of the sea salt containers is a closed packet and the sea salt material is pure sea salt. In the salt adding step, the user tears open the packet before emptying the pure sea salt into the water container.

According to some embodiments, the water container comprises a lid and the water container is shaped to define an opening. After the water filling step and the salt adding step, the lid selectively securely closes and seals the opening and, in the alternative, the user selectively pours or rinses with the predetermined volume of water through the opening.

According to some embodiments, in the water filling step, the predetermined temperature falls within a temperature range of between about tepid temperature and about room temperature.

According to some embodiments, in the water filling step, the predetermined volume of water falls within a volume range of between about 0.18 liters (6 ounces) and about 1.5 liters (50.7 ounces) and, more particularly, between about 0.40 liters (13.5 ounces) and about 0.75 liters (25.4 ounces). The predetermined volume of water may be between about 0.41 liters (14.0 ounces) and about 0.47 liters (16.0 ounces).

According to some embodiments, in the water filling step, the predetermined water pressure and pH may fall within normal ranges for distilled and tap waters consumed by humans.

According to some embodiments, there is provide a method for a sea salt mouth rinse kit comprising a bottle with a fill line; and a plurality of packets, each packet containing a predetermined quantity of pure sea salt, the fill line containing a predetermined volume of water proportional to the predetermined quantity of pure sea salt, the method comprising a water filling step for filling the bottle with water to the fill line, such that the bottle then contains the predetermined volume of water, and a salt adding step for adding one of the packets to the bottle, wherein the predetermined quantity of pure sea salt fully dissolves and closely approaches full saturation, without precipitating out, in the predetermined volume of water at a water pressure and pH both falling within normal ranges for distilled water, the water releasing through an opening in the bottle, and a cap selectively securely closes and seals the opening.

In some embodiments, the method involves dissolving the predetermined quantity of the sea salt material in water to a predetermined saturation, without precipitating out, in the predetermined volume of water at a predetermined temperature, and at a predetermined water pressure and pH.

In some embodiments, each of the sea salt containers is a closed packet which may be selectively torn open, and the sea salt material is pure sea salt.

In some embodiments, the water container comprises a lid and, releasing, by an opening defined in the water container, the predetermined volume of water when the container is filled, the lid can be selectively used to securely close and seal the opening.

In some embodiments, the predetermined volume of water falls within a volume range of selected from: between about 0.18 liters (6 ounces) and about 1.5 liters (50.7 ounces), between about 0.40 liters (13.5 ounces) or about 0.75 liters (25.4 ounces), and between about 0.41 liters (14.0 ounces) and about 0.47 liters (16.0 ounces).

In some embodiments, the water container is a bottle, and the lid is a cap.

In another aspect, embodiments provide a sea salt mouth rinse kit comprising a water container that enclose one or more sea salt containers, wherein the water container has a fill line to contain a predetermined volume of water and at a region where the water container opens, each of the sea salt containers contains a predetermined quantity of a sea salt material, the predetermined volume of water proportional to the predetermined quantity of sea salt material.

In some embodiments, the predetermined quantity of the sea salt material fully dissolves to a predetermined saturation, without precipitating out, in the predetermined volume of water at a predetermined temperature, and at a predetermined water pressure and pH.

In some embodiments, each of the sea salt containers is a closed packet which may be selectively torn open, and the sea salt material is pure sea salt.

In some embodiments, the water container comprises a lid and the water container is shaped to define an opening, the opening to permit release of the predetermined volume of water when the container is filled, the lid can be selectively used to securely close and seal the opening.

In some embodiments, the predetermined volume of water falls within a volume range of selected from: between about 0.18 liters (6 ounces) and about 1.5 liters (50.7 ounces), between about 0.40 liters (13.5 ounces) or about 0.75 liters (25.4 ounces), and between about 0.41 liters (14.0 ounces) and about 0.47 liters (16.0 ounces).

In some embodiments, the water container is a bottle, and the lid is a cap.

In another aspect, there is provide a sea salt mouth rinse kit that includes: a bottle with a fill line; and a plurality of packets, each containing a predetermined quantity of pure sea salt, the fill line containing a predetermined volume of water proportional to the predetermined quantity of pure sea salt for full saturation.

In some embodiments, the predetermined quantity of the sea salt material is adapted to fully dissolve to a predetermined saturation, without precipitating out, in the predetermined volume of water at a predetermined temperature, and at a predetermined water pressure and pH.

In some embodiments, each of the sea salt containers is a closed packet which may be selectively torn open, and the sea salt material is pure sea salt.

In some embodiments, the water container comprises a lid and the water container is shaped to define an opening, the opening to permit release of the predetermined volume of water when the container is filled, the lid can be selectively used to securely close and seal the opening.

In some embodiments, the predetermined volume of water falls within a volume range of selected from: between about 0.18 liters (6 ounces) and about 1.5 liters (50.7 ounces), between about 0.40 liters (13.5 ounces) or about 0.75 liters (25.4 ounces), and between about 0.41 liters (14.0 ounces) and about 0.47 liters (16.0 ounces).

In some embodiments, the water container is a bottle, and the lid is a cap.

In some embodiments, the bottle opens at fill line to release the salt packets.

In some embodiments, the water container opens at fill line to release the salt packets.

Other advantages, features and characteristics of the various embodiments, as well as methods of operation and functions of the related elements of the structure, and the combination of parts and economies of manufacture, will become more apparent upon consideration of the following detailed description with reference to the accompanying figures, the latter of which are briefly described hereinbelow.

### BRIEF DESCRIPTION OF THE FIGURES

The novel features which are believed to be characteristic of the sea salt mouth rinse kit and method of using same according to the present invention, as to its structure, organization, use and method of operation, together with further objectives and advantages thereof, will be better understood from the following figures by way of example. It is expressly understood, however, that the figures are for the purpose of illustration and description only, and are not intended as a definition of the limits of the invention. In the accompanying figures:

FIG. 1 is a photograph of a bottle included in the kit according to an embodiment of the invention, shown with the bottle in an assembled configuration;

FIG. 2 is a photograph of the bottle of FIG. 1, along with two sea salt packets included in the kit according to an embodiment of the invention;

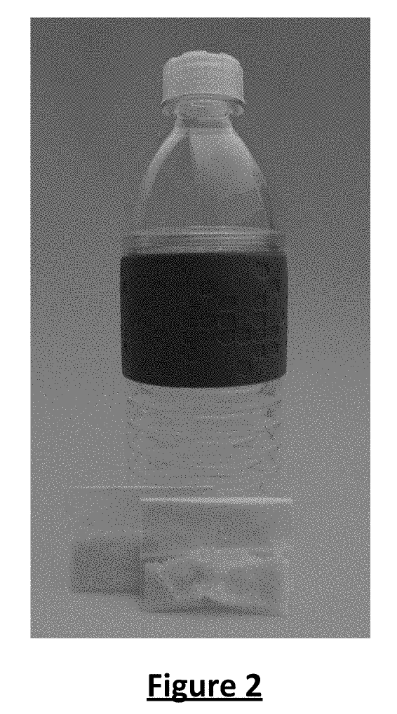

FIG. 3 is a photograph of the bottle and sea salt packets of FIG. 2, shown with the bottle both in an assembled configuration and in a partially disassembled configuration;

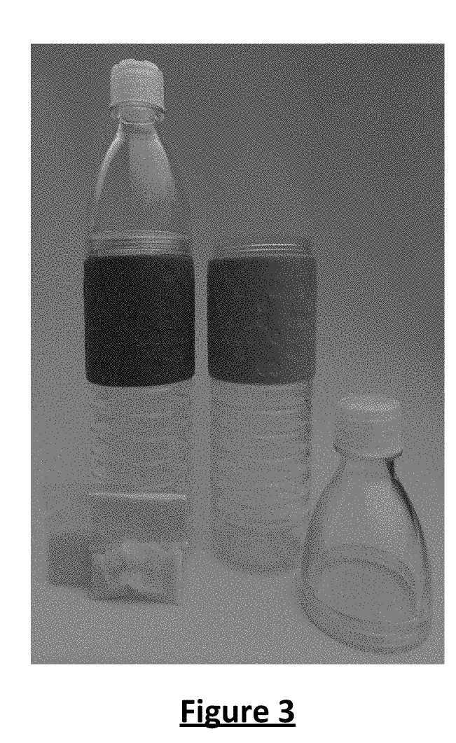

FIG. 4 is a photograph of the bottle of FIG. 1, along with a plurality of sea salt packets included in the kit, inside and outside the bottle, according to another embodiment of the invention;

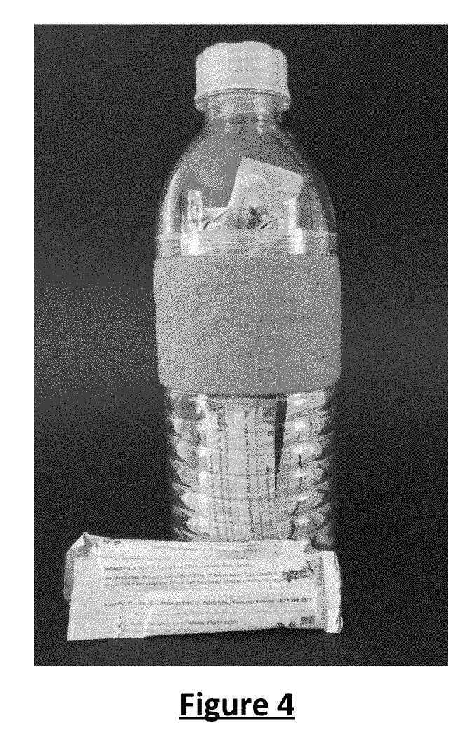

FIG. 5 is a photograph of the bottle and sea salt packets of FIG. 4, shown with the bottle both in an assembled configuration and in a partially disassembled configuration;

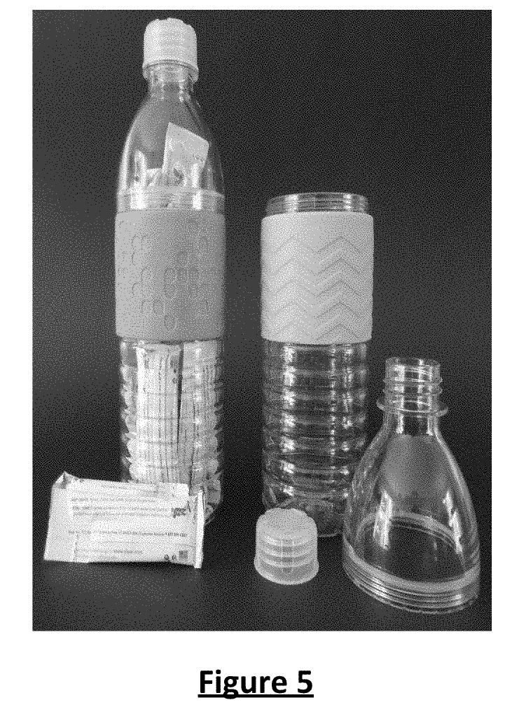

FIG. 6 is a photograph of three bottles according to FIG. 1, showing a range of different sea salt packets included in the kit, inside and outside the bottle, according to various embodiments of the invention;

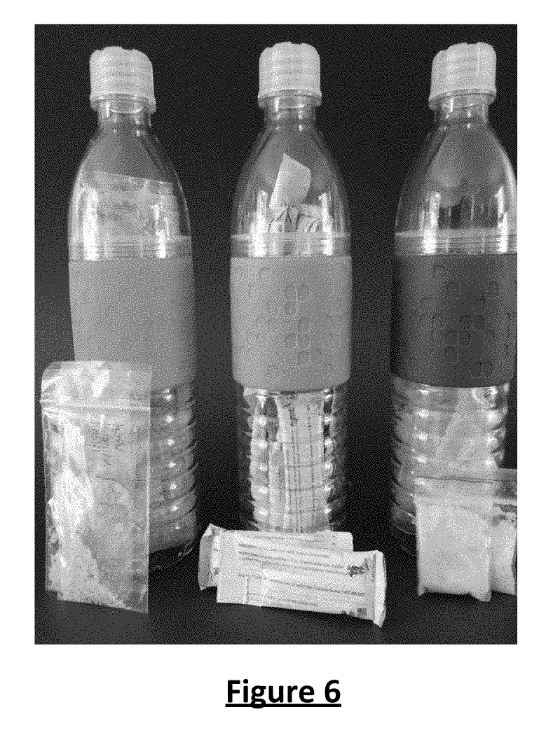

FIG. 7 is a photograph of a different bottle along with a plurality of sea salt packets included, inside the bottle, in a smaller compact kit according to yet another embodiment of the invention, shown with the bottle in a partially disassembled configuration;

FIG. 8 is a photograph of a different bottle for a kit according to another embodiment of the invention;

FIG. 9 is a photograph of a different bottle for a kit according to another embodiment of the invention;

FIG. 10 is a photograph of bottles similar to those shown in FIG. 1 for a kit according to another embodiment of the invention;

FIG. 11 is a photograph of a different bottle for a kit according to another embodiment of the invention;

FIG. 12 is a photograph of a different bottle for a kit according to another embodiment of the invention;

FIG. 13 is a photograph of a different bottle for a kit according to another embodiment of the invention;

FIG. 14 is a photograph of a different bottle for a kit according to another embodiment of the invention; and

FIG. 15 is a photograph of a plurality of sea salt packets for a kit according to another embodiment of the invention.

### DETAILED DESCRIPTION

In the accompanying figures, there is shown an example embodiment of a sea salt mouth rinse kit. It includes a water bottle. The bottle has a fill line. When filled to the fill line, the bottle contains a predetermined volume of water, such as between about 0.41 liters (14.0 ounces) and about 0.47 liters (16.0 ounces) according to some embodiments. The bottle includes a cap. The bottle is also shaped to define an opening. A user may selectively pour or rinse with the water through the opening after the container has been filled. The cap can be selectively used to securely close and seal the opening.

In some embodiments, the bottle may include three (3) pieces: a bottom portion, a top portion, and the cap. Preferably, all three (3) pieces may be screwed together to afford a complete leak proof seal. Preferably, a three (3) piece bottle of this general type may be easier to clean.

In some embodiments, the bottle may, for example, be the reusable bottle that which may be readily filled and re-filled and washed between uses, without ending up in a landfill. A handy reusable bottle of this general type may have a twist-open body to readily add the sea salt packets. It also may have a leak-proof easy-off lid, and a colorful soft grip band to facilitate its secure handling.

The bottle can be 100% free of any Bisphenol A (BPA) and other harmful chemicals which should be avoided in products intended for everyday use.

The bottle will not be composed of metals (e.g. stainless-steel) as the metals may undergo unintended corrosion with the salt.

The bottle may not be composed of glass parts which may be more prone to breaking.

The sea salt mouth rinse kit according to the invention additionally includes a plurality of closed packets. Each of the packets may be selectively torn open and contains a predetermined quantity of pure sea salt. The pure sea salt may be a natural light gray salt which come from a brine and/or a “mother liquor” and contains one or more minerals useful by the human body. It will also be free of pesticides, herbicides, and harmful chemicals, and guaranteed to have no additives.

According to embodiments, the predetermined quantity of pure sea salt is adapted to fully dissolve and closely approach full saturation, without precipitating out, in the predetermined volume of water at a tepid or room temperature, and at a water pressure and pH both falling within normal ranges for distilled water consumed by humans.

More particularly, according to embodiments, each of the sea salt packets contains about 1 teaspoon (between about 5.0 grams and about 6.0 grams) of pure sea salt.

More particularly, according to some embodiments, each sea salt packet can be about 9.0 cm in width and about 1.50 cm in height.

The kit can include a bottle with a plurality of the closed salt packets inside, and a number of additional salt packets (e.g., perhaps totaling about thirty packets), along with a handy instruction sheet inside a box. The box may be a reverse tuck carton or a locking bottom carton which has been custom pre-printed with information useful to the user.

A method of using the sea salt mouth rinse kit includes a water filling step and a salt adding step. In the water filling step, the user fills the bottle with water to the fill line, such that it then contains the predetermined volume of water, between about 0.41 liters (14.0 ounces) and about 0.47 liters (16.0 ounces) according to embodiments, at a tepid or room temperature, and at a water pressure and pH both falling within normal ranges for distilled water consumed by humans.

In the salt adding step, the user tears opens and empties one of the packets into the bottle, such that the predetermined quantity of pure sea salt fully dissolves and closely approaches full saturation, without precipitating out, in the predetermined volume of water.

After the water filling step and the salt adding step, the cap selectively securely closes and seals the bottle's opening and, in the alternative, the user selectively pours or rinses with the water through the opening.

The sea salt mouth rinse kit and method of using same afford a convenient all-in-one package that makes it easier for those in a vulnerable health situation to quickly make this a very important part of their healing journey.

In some embodiments, the user pours 1 packet of pure sea salt into the bottle filled with between about 0.41 liters (14.0 ounces) and about 0.47 liters (16.0 ounces) of warm or room temperature distilled water. The user then closes the bottle, making sure it is sealed at both the middle of the bottle and the cap, and shakes the bottle to better mix and dissolve the salt. The user may carry out this process many times throughout the day and, in turn, gently swishing the mouth with the solution in the bottle before spitting it out. The user may perhaps carry out this process at least 4 or more times each day, e.g., every 1 to 2 hours as needed.

The user will discuss with a doctor their contemplated use of the sea salt mouth rinse kit according to the invention. For a sore throat, the user may gargle with the sea salt solution 5-7 times throughout the day, spitting out the solution. Chemotherapy patients may swish the sea salt solution gently in the mouth throughout the day, spitting it out each time, and the entire bottle may be used each day, while progressing through chemotherapy treatments. Dental patients may ask their dentists if use of the sea salt mouth rinse kit, according to the invention, may be right for them. Generally, a dental patient may be able to start rinsing very gently 24 hours after treatment, spitting out the solution after each rinse. In all cases, use should be discontinued if any adverse reaction occurs.

The bottle may be cleaned daily. The three parts of the bottle may be completely separated from one another. The lower and upper parts of the bottle may be safely and completely cleaned in a dishwasher or by hand. The cap may be hand-washed separately.

Use of the sea salt mouth rinse kit may afford one or more of the following advantages to the user:
it may help to inhibit the formation of mouth blisters which can be painful (e.g., for chemotherapy patients).
gargling may help to alleviate, reduce or minimize pain and/or inflammation of tissues (e.g., for sore throats).
it may help to clean sites within the mouth, to soothe mucosal tissues, and/or to promote recovery (e.g., for dental patients, oral surgery patients, orthodontic patients, or periodontal patients).

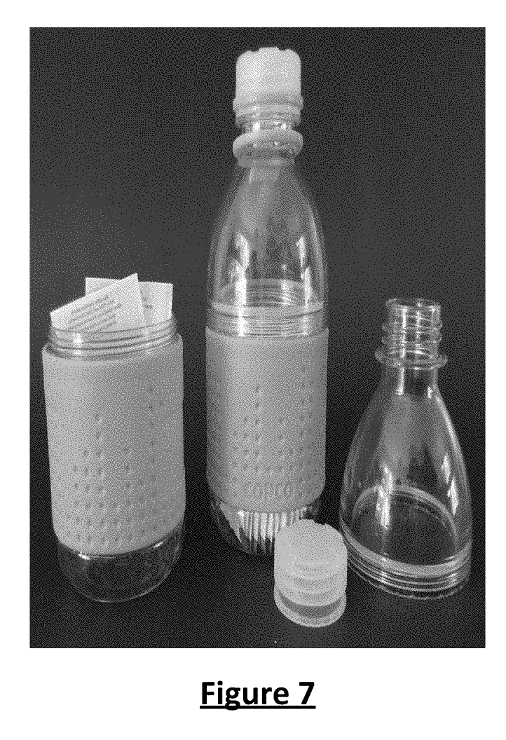

According to some embodiments, the predetermined volume of water is about 0.18 liters (7 ounces), each of the sea salt packets contains about ½ teaspoon (about 2.75 grams) of pure sea salt, and the user pours 1 packet of pure sea salt into the bottle filled with 0.18 liters (7 ounces) of warm or room temperature distilled water. A smaller compact version of the kit which then might be supplied in this respect, as shown in FIG. 7, may find particular utility by people on-the-go and/or who wish to carry the kit in their purse, to be used during the day such as, for example, at work.

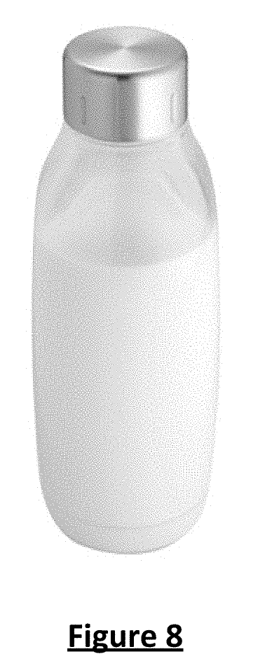

FIG. 8 is a photograph of a different bottle for a kit according to another embodiment of the invention. The bottle contains liquid and is air-tight and leak-proof, dishwasher safe. The bottle is made of clear, durable BPA free plastic with a brushed stainless steel lid.

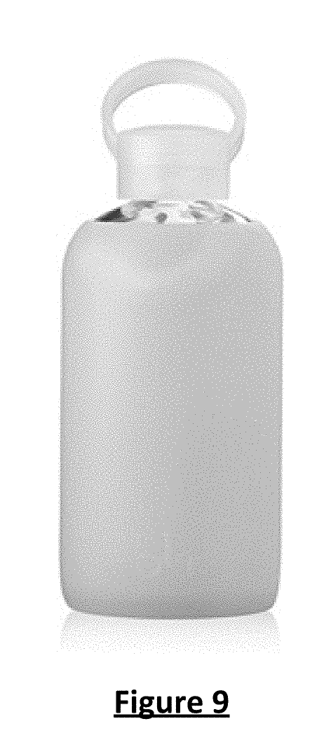

FIG. 9 is a photograph of a different bottle for a kit according to another embodiment of the invention. The bottle contains 500 ml of solution. The bottle has a soft, grippy silicone sleeve that adds color, insulates hot or cold liquid, allows for single-handed use, keeps it quiet when set down. The cap is leak-proof, designed to thread perfectly through the fingers to show off your bottle. The shape includes a small mouth opening allows for easy, no-spill sips, and its wide base is tip-resistant. The glass bottle is clean and pure, and does not leave a metal or plastic taste and does not leach chemicals when sitting in the car. Its BPA-free, phthalate-free food-grade polypropylene plastic and silicone cap's design protects the mouth even if you place the cap on a table or counter. And staying optimally hydrated keeps you in good health. The bottle fit most cup holders, accommodates smaller ice and is dishwasher safe.

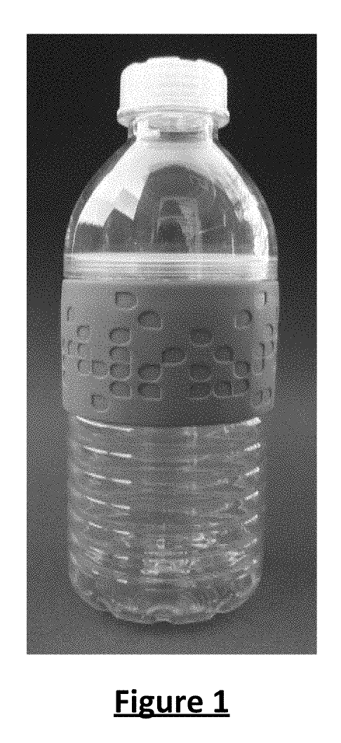

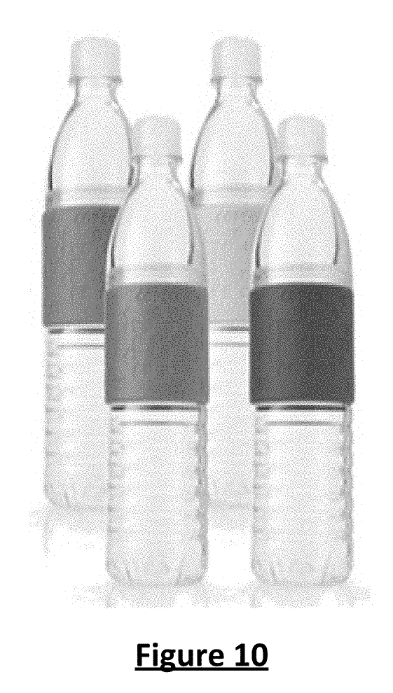

FIG. 10 is a photograph of bottles similar to those shown in FIG. 1 for a kit according to another embodiment of the invention. This handy reusable bottle has a twist open body to add contents of the sea salt package. It can be made from durable Tritan and is 100% BPA free. The double wall insulation keeps drinks the preferred temperature longer. The bottle has a 16 ounce capacity and a threaded on lid with flip up straw, in some embodiments. It is dishwasher safe. The bottle has 3 pieces (2 body pieces and a cap). The bottle is easy to clean, easy to fill, can be packaged in 2 parts in the kit box to reduce the required shelf space, its esthetically pleasing, BPA free, and the silicone grip makes it easy to hold.

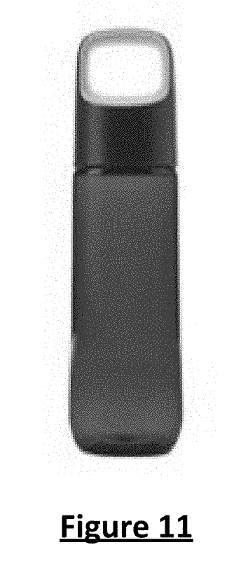

FIG. 11 is a photograph of a different bottle for a kit according to another embodiment of the invention. The body can be made from Tristan material and the cap can be polyproplyne. The bottle sleeve can be food grade silicon. The bottle can have a 500 ml capacity. The twist off cap shields the mouth piece from dirt.

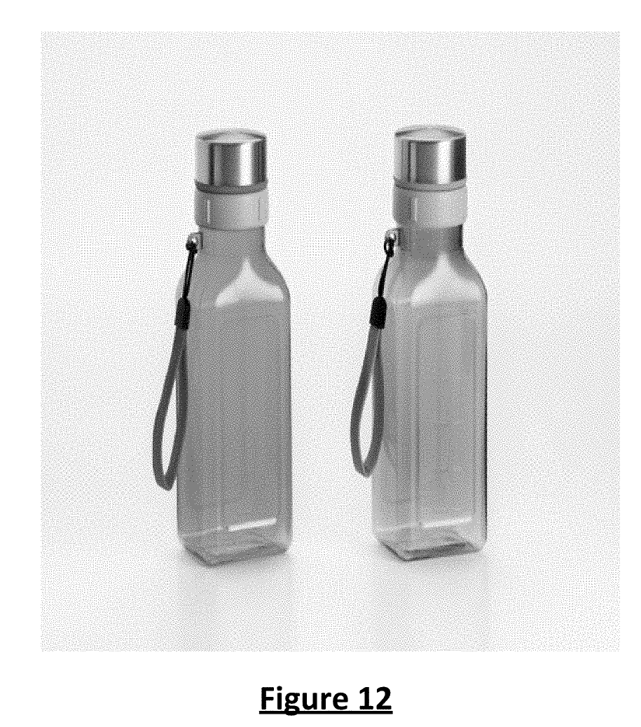

FIG. 12 is a photograph of a different bottle for a kit according to another embodiment of the invention. The rectangular designed Tritan plastic bottle can hold up to 24 oz in solution. The bottle comes with a measuring gauge on the back to track how much of your drink is left and how much water to add.

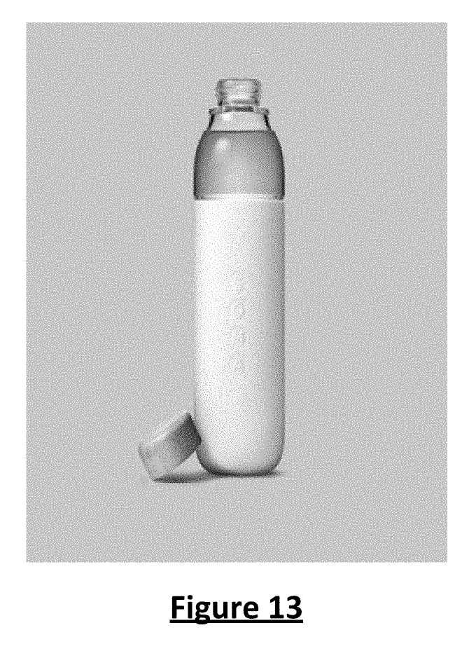

FIG. 13 is a photograph of a different bottle for a kit according to another embodiment of the invention. The glass water bottle is lightweight and durable, holds 17 oz. and features a soft silicone sleeve. The natural bamboo cap is leak-proof and made from a renewable resource. The smooth mouth design delivers a comfortable drinking experience.

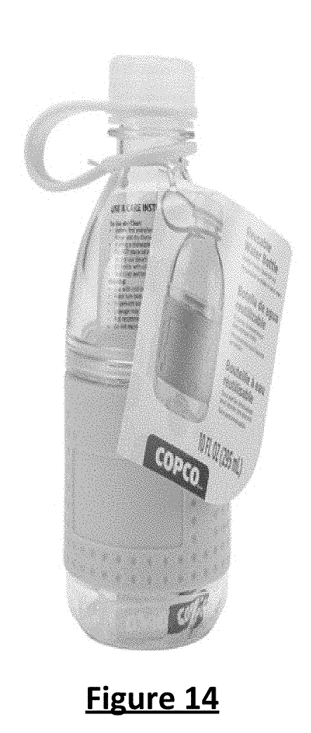

FIG. 14 is a photograph of a different bottle for a kit according to another embodiment of the invention. The bottle can be 10-Fluid-Ounce (295 ml) for example.

The bottle can be made of different material including plastic, glass and stainless steel. Glass is BPA free and easy to clean.

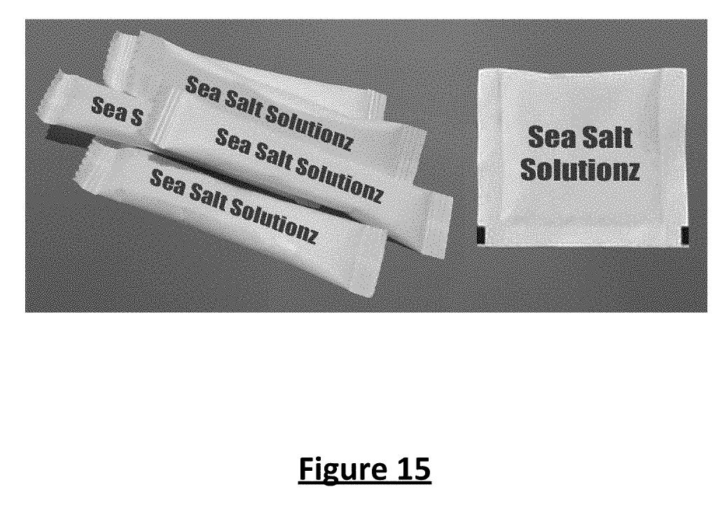

FIG. 15 is a photograph of a plurality of sea salt packets for a kit according to another embodiment of the invention. The packet can be approximately 6 cm×4 cm (2 2/16 in×1 9/16 in). The tube packet can be approximately 9 cm×1.50 cm (3 8/16 in× 10/16 in.) The packet or tube is easy to use and provides a specific amount of sea salt that works with the fill-line of the container to bottle to add the specific amount of sea salt to a specific amount of distilled water in a BPA free bottle. The packet is convenient, simple, easy to open, easy to pour and easy to store.

The invention is contemplated for use in association with mouth rinsing and sea salt, to afford increased functionality and/or advantageous utilities in association with same. The invention, however, is not so limited.

The foregoing description has been presented for the purpose of illustration and is not intended to be exhaustive or to limit the invention to the precise form disclosed.

Naturally, in view of the teachings and disclosures herein, persons having ordinary skill in the art may appreciate that alternate designs and/or embodiments of the invention may be possible (e.g., with substitution of one or more components for others, with alternate configurations of components, etc.). Although some of the components, relations, configurations and/or steps according to the invention are not specifically referenced in association with one another, they may be used, and/or adapted for use, in association therewith. All of the aforementioned, depicted and various structures, configurations, relationships, utilities and the like may be, but are not necessarily, incorporated into and/or achieved by the invention. Any one or more of the aforementioned structures, configurations, relationships, utilities and the like may be implemented in and/or by the invention, on their own, and/or without reference, regard or likewise implementation of any of the other aforementioned structures, configurations, relationships, utilities and the like, in various permutations and combinations.

Other modifications and alterations may be used in the design, manufacture, and/or implementation of other embodiments according to the present invention.

1. A method for a sea salt mouth rinse kit comprising a bottle with a fill line; and a plurality of packets, each packet containing a predetermined quantity of pure sea salt, the fill line containing a predetermined volume of water proportional to the predetermined quantity of pure sea salt, the method comprising a water filling step for filling the bottle with water to the fill line, such that the bottle then contains the predetermined volume of water, and a salt adding step for adding one of the packets to the bottle, wherein the predetermined quantity of pure sea salt fully dissolves and closely approaches full saturation, without precipitating out, in the predetermined volume of water at a water pressure and pH both falling within normal ranges for distilled water, the water releasing through an opening in the bottle, and a cap selectively securely closes and seals the opening.

2. The method of claim 1, comprising dissolving the predetermined quantity of the sea salt material in water to a predetermined saturation, without precipitating out, in the predetermined volume of water at a predetermined temperature, and at a predetermined water pressure and pH.

3. The method of claim 1, wherein each of the sea salt containers is a closed packet which may be selectively torn open, and the sea salt material is pure sea salt.

4. The method of claim 1, wherein the water container comprises a lid and, releasing, by an opening defined in the water container, the predetermined volume of water when the container is filled, the lid can be selectively used to securely close and seal the opening.

5. The method of claim 1, wherein the predetermined volume of water falls within a volume range of selected from: between about 0.18 liters (6 ounces) and about 1.5 liters (50.7 ounces), between about 0.40 liters (13.5 ounces) or about 0.75 liters (25.4 ounces), and between about 0.41 liters (14.0 ounces) and about 0.47 liters (16.0 ounces).

6. The method of claim 1, wherein the water container is a bottle, and the lid is a cap.

7. A sea salt mouth rinse kit comprising a water container that enclose one or more sea salt containers, wherein the water container has a fill line to contain a predetermined volume of water and at a region where the water container opens, each of the sea salt containers contains a predetermined quantity of a sea salt material, the predetermined volume of water proportional to the predetermined quantity of sea salt material.

8. The sea salt mouth rinse kit of claim 7 wherein the predetermined quantity of the sea salt material fully dissolves to a predetermined saturation, without precipitating out, in the predetermined volume of water at a predetermined temperature, and at a predetermined water pressure and pH.

9. The sea salt mouth rinse kit of claim 7 wherein each of the sea salt containers is a closed packet which may be selectively torn open, and the sea salt material is pure sea salt.

10. The sea salt mouth rinse kit of claim 7 wherein the water container comprises a lid and the water container is shaped to define an opening, the opening to permit release of the predetermined volume of water when the container is filled, the lid can be selectively used to securely close and seal the opening.

11. The sea salt mouth rinse kit of claim 7 wherein the predetermined volume of water falls within a volume range of selected from: between about 0.18 liters (6 ounces) and about 1.5 liters (50.7 ounces), between about 0.40 liters (13.5 ounces) or about 0.75 liters (25.4 ounces), and between about 0.41 liters (14.0 ounces) and about 0.47 liters (16.0 ounces).

12. The sea salt mouth rinse kit of claim 7 wherein the water container is a bottle, and the lid is a cap.

13. A sea salt mouth rinse kit includes: a bottle with a fill line; and a plurality of packets, each containing a predetermined quantity of pure sea salt, the fill line containing a predetermined volume of water proportional to the predetermined quantity of pure sea salt for full saturation.

14. The sea salt mouth rinse kit of claim 13 wherein the predetermined quantity of the sea salt material is adapted to fully dissolve to a predetermined saturation, without precipitating out, in the predetermined volume of water at a predetermined temperature, and at a predetermined water pressure and pH.

15. The sea salt mouth rinse kit of claim 13 wherein each of the sea salt containers is a closed packet which may be selectively torn open, and the sea salt material is pure sea salt.

16. The sea salt mouth rinse kit of claim 13 wherein the water container comprises a lid and the water container is shaped to define an opening, the opening to permit release of the predetermined volume of water when the container is filled, the lid can be selectively used to securely close and seal the opening.

17. The sea salt mouth rinse kit of claim 13 wherein the predetermined volume of water falls within a volume range of selected from: between about 0.18 liters (6 ounces) and about 1.5 liters (50.7 ounces), between about 0.40 liters (13.5 ounces) or about 0.75 liters (25.4 ounces), and between about 0.41 liters (14.0 ounces) and about 0.47 liters (16.0 ounces).

18. The sea salt mouth rinse kit of claim 13 wherein the water container is a bottle, and the lid is a cap.

19. The sea salt mouth rinse kit of claim 18 wherein the bottle opens at fill line to release the salt packets.

20. The sea salt mouth rinse kit of claim 13 wherein the water container opens at fill line to release the salt packets.
